(19) United States
(12) Patent Application Publication
(10) Pub. No.: US 2026/0311864 A1
(43) Pub. Date: Oct. 8, 2026

(54) REAL-TIME REMOTE GUIDANCE

(71) Applicant: Exo Imaging, Inc., Santa Clara, CA (US)

(72) Inventors: Saniya SALIM, San Francisco, CA (US); Eric TWARDZICKI, San Francisco, CA (US); Sabina KUGEL, Belmont, CA (US); Michael PUPO, Phoenix, AZ (US)

(21) Appl. No.: 19/481,796

(22) PCT Filed: May 3, 2024
(86) PCT No.: PCT/US2024/027876

§ 371 Date: Nov. 5, 2025

Related U.S. Application Data
(60) Provisional application No. 63/464,431, filed on May 5, 2023.

(51) Int. Cl. G16H 40/67; A61B 34/20

(52) U.S. Cl. CPC G16H 40/67; A61B 34/20

(57) ABSTRACT

A method for providing remote guidance from a skilled resource to a local user during a medical procedure includes transmitting information about the procedure from the local user to the skilled resource and allowing the skilled resource to provide real-time feedback to the local user and/or take remote control of the local user's medical device.

### CROSS-REFERENCE TO RELATED APPLICATIONS

This application claims the benefit of U.S. Provisional Application No. 63/464,431, filed May 5, 2023, the entirety of which is incorporated herein by reference.

### FIELD OF THE INVENTIONS

The present disclosure generally relates to systems, methods, and devices for operating, guiding, or controlling a medical device, such as an ultrasound probe, via real-time remote interaction.

### BACKGROUND

Ultrasound imaging is an imaging method that uses sound waves to produce images of structures within a patient's body. Because ultrasound images are captured in real-time, they can also show movement of the body's internal organs as well as blood flowing through the blood vessels. The images can provide valuable information for diagnosing and directing treatment for a variety of diseases and conditions.

### SUMMARY

The systems, methods and devices of this disclosure each have several innovative aspects, no single one of which is solely responsible for the desirable attributes disclosed herein.

Portable (e.g., handheld, and/or battery-operated) ultrasound devices provide greater use flexibility than traditional larger-scale or wired ultrasound devices. However, in accordance with at least some embodiments disclosed herein is the realization that newer, portable ultrasound devices require training to properly use the device. When a novice ultrasound device user has questions about how to operate the device or perform a medical procedure, including successfully completing one or more ultrasound scans, the novice can benefit from the perspective of more experience or a more expansive knowledge set. This greater knowledge and/or experience could come from a more experienced user, such as a doctor, nurse, skilled technician, or other medical professional or person skilled in the use of the device. This greater knowledge and/or experience could also come in the form of artificial intelligence (AI), generative artificial intelligence, a virtual assistant, or other computer or virtual system that can provide guidance to a user. The innovative principles disclosed herein, in accordance with some embodiments, can be applied generally to telehealth or telemedicine capabilities in order to allow a novice user to interact with a more experienced user or system through a remote, electronic, or online connection in order for the novice user to receive guidance and feedback from the experienced user.

The present disclosure provides a system and related methods whereby a novice user of an ultrasound device can receive feedback and other information about the device, the procedure, the methods used, the results, and other factors while using the ultrasound device to conduct a procedure. The feedback can be provided by a more experienced user or a knowledgeable artificial intelligence or other virtual module. Optionally, the feedback interaction can be provided in real time and enable the novice user to perform the medical procedure leveraging a skill set and expertise that is greater than their own.

As disclosed herein, in some embodiments, a method for providing remote guidance from an experienced user to a more novice user during a medical procedure comprises transmitting information about the procedure from the novice user to the experienced user or system, allowing the experienced user or system to provide feedback or interact with the user or device during or regarding the procedure, and communicating that feedback or interaction from the experienced user or system to the novice user. It should be understood herein that any reference to the experienced user or to the experienced system may refer to a more experienced physical user, such as a doctor, nurse, skilled technician, or other medical professional or person skilled in the use of the device or also to a form of artificial intelligence (AI), generative artificial intelligence, a virtual assistant, or other computer or virtual system.

As disclosed herein, in some embodiments, the novice user may use an electronic device to video themself performing the medical procedure and provide that video to the experienced user. The experienced user can then view how the novice user is performing the procedure and provide their feedback. In some embodiments, this video is transmitted via a live stream or in real-time, such that the experienced user can guide the more novice user in how to perform the medical procedure.

Optionally, in accordance with some embodiments, the video can be provided to more than one person other than the experienced user, such as to a panel of clinicians for educational and/or medical purposes.

As disclosed herein, in some embodiments, the medical procedure can be any of a variety of medical procedures. In particular, some embodiments contemplate the performance of the method using an ultrasonic scan via a handheld ultrasonic imaging probe. Further, additional devices, such as a transmitter and/or a mobile device or smartphone, including traditional mobile phones, can be utilized in the systems and methods disclosed herein.

In some embodiments, the novice user and the experienced user both have electronic devices, such as smartphones or tablets, onto which an application is downloaded. The application can serve as an interface or platform that can allow the novice user's device to communicate with the experienced user's device, conveying or transmitting video, screen, and/or audio communication, as well as to permit the experienced user to provide feedback to the novice user. For example, the feedback from the experienced user can be provided in real time, during performance of the medical procedure. In addition to conveying or transmitting feedback to the novice user, the application may optionally permit the experienced user to remotely control one or more settings and/or features of the medical device (e.g., ultrasonic probe).

In implementing some embodiments of the methods and systems disclosed herein, a novice user with little or no formal training or experience in the use of a given medical device or medical procedure can receive guidance and benefit from the expertise of another individual or group in performing a medical procedure. For example, even experienced clinicians may at times need guidance on how to operate a medical device in order to complete a medical procedure. Indeed, especially when using new medical devices, a user of the medical device can benefit from guidance on how to program various settings relating to the medical device. Further, the user of the medical device may benefit from guidance on where on a patient to begin a diagnostic scan. Moreover, along with these and other realizations that are the subject of the present disclosure, a clinician can benefit from guidance related to non-medical explanations, workflows, or other processes, including processes relevant to insurance billing or reimbursement strategies, client documentation, and evidence collection that may be associated with or necessary for certain careers and professional responsibilities.

In some embodiments, the experienced user can provide feedback via transmitting sound, drawing notation on the screen of their device, by the use of various pre-set graphical overlays which they can control, and/or by showing a video of themself performing the desired action. Controls may be available on the display screen to allow the experienced user to change the size, direction, or other attributes of the overlay.

In some embodiments, the experienced user can control various settings or features of the medical device through the shared application. For example, when using an ultrasonic probe, these settings and features can include mode selection, measurements (such as volume or velocity), calculations, adjustments to presets, brightness, scan settings (such as gain, scan depth, size/zoom, color, focus, or position), image and video capture, image freeze, annotation and capture tools, power settings, software settings, tool selection, share and data transfer settings and controls, and/or various other features and controls as may be implemented for a given medical device.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 shows a schematic diagram of a communication system, according to some embodiments of the present disclosure.

FIG. 2 shows a schematic diagram of an ultrasonic imager, according to some embodiments of the present disclosure.

FIG. 3 shows an example electronic device display screen displaying selection options, according to some embodiments of the present disclosure.

FIG. 4 shows an example call notification and device display screen, according to some embodiments of the present disclosure.

FIGS. 5A-5D show an example device display screen with feedback options, according to some embodiments of the present disclosure.

FIGS. 6A-6D show an example device display screen illustrating one feedback option, according to some embodiments of the present disclosure.

FIGS. 7A-7F show an example device display screen illustrating a second feedback option, according to some embodiments of the present disclosure.

FIGS. 8A-8F show an example device display screen illustrating a third feedback option, according to some embodiments of the present disclosure.

FIGS. 9A-9D show an example device display screen illustrating a fourth feedback option, according to some embodiments of the present disclosure.

FIG. 10A shows an example device display screen with drawing overlay options, according to some embodiments of the present disclosure.

FIG. 10B shows an example device display screen illustrating a drawn overlay, according to some embodiments of the present disclosure.

FIG. 10C shows an example device display screen with feedback options, according to some embodiments of the present disclosure.

FIG. 10D shows an example device display screen with a drawn overlay, according to some embodiments of the present disclosure.

FIG. 10E shows an example device display screen with image capture controls, according to some embodiments of the present disclosure.

FIG. 11A shows an example device display screen with notifications, according to some embodiments of the present disclosure.

FIG. 11B shows an example device display screen with a toggled view, according to some embodiments of the present disclosure.

FIG. 11C shows example screen notifications, according to some embodiments of the present disclosure.

### DETAILED DESCRIPTION

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for ease of understanding.

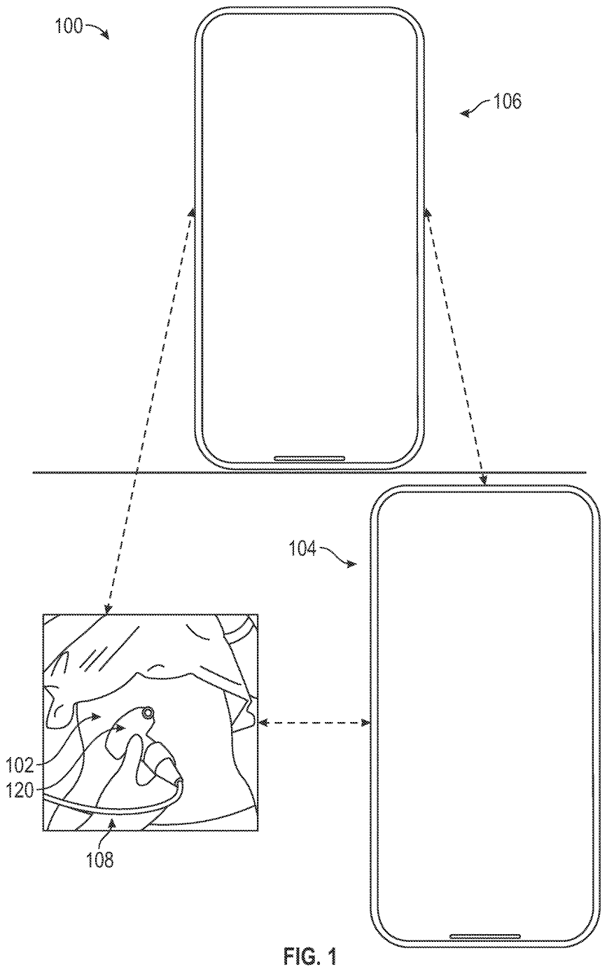

FIG. 1 is a diagram illustrating the communication system 100, according to some embodiments of the present disclosure. The system 100 may be used for a patient 102 undergoing a medical procedure involving a medical device, such as an ultrasound probe 120. The probe 120 may be operated by an on-site device user 108 to perform an examination of the patient 102. In some embodiments, the device user 108 may also use and operate a local electronic device 104 in conjunction with use of the probe 120. The local electronic device 104 can be in electronic communication with the probe 120 and receive and/or provide instructions to the probe 120.

In accordance with some embodiments, the local electronic device 104 may be used to video the medical procedure as it is performed on the patient 102 by the probe 120. The on-site user 108 can use a first hand to control the probe 120 and a second hand to operate the local electronic device 104 and record or transmit a live view of the procedure as it is being performed on the patient 102.

For example, the local electronic device 104 may be operable to call a second, remote electronic device 106. The remote electronic device 106 may be operated by a second, remote user, who can witness the procedure as it is being performed. In some embodiments, the remote electronic device 106 can be aided by, comprise, or include all or part of an AI or other computerized system. In accordance with some embodiments, the electronic devices 104, 106, as well as any other electronic devices that may be in communication with at least the local electronic device 104 and/or the remote electronic device 106, can include mobile phones (i.e., smart phones), tablets, display screens (such as smart TVs and other smart screens), as well as other display or non-display communication devices that can communicate (i.e., sending and/or receiving sound, image, video, or other data to and/or from the electronic device 104). Additionally, the system 100 may include multiple local electronic devices 104 and multiple remote electronic devices 106.

As discussed herein, the present disclosure advantageously provides a unique system by which the user 108 can broadcast or share the procedure with the remote user(s), who can be a skilled professional or otherwise skilled in the operation of the medical device or that may join for observational purposes. Through the broadcast and observation, the skilled professional can contribute expertise and/or learn as the user 108 conducts the procedure. As such, a team of two or more physicians can observe, interact, instruct, or otherwise collaborate to improve the performance of the procedure by the user 108. As discussed herein, this incredible system can revolutionize the way physicians and home-care consumers achieve better imaging results.

In some embodiments, the local electronic device 104 can be operated by the on-site user 108 using a first hand, and the on-site user 108 may use the probe 120 with the other hand. However, the local electronic device 104 can also be stationary, such as a computer, television, or projection screen, which thereby enables the on-site user 108 to use one or both hands to manipulate the medical device or probe 120. Accordingly, the local electronic device 104 may be operated by the on-site user 108, a person other than the on-site user 108, or may be able to operate without human interaction.

The operator of the remote electronic device 106 may be remote from the on-site user 108. The on-site user 108 and the operator of the remote electronic device 106 may be physically separate such that communication through the local electronic device 104 and the remote electronic device 106 is more convenient, faster, or easier than the operator of the remote electronic device 106 providing in-person feedback to the on-site user 108. This remote connection may allow the on-site user 108 to receive feedback from a wider variety of sources than if the on-site user 108 could only receive feedback from, for example, physicians or other skilled users of the probe 120 who could provide guidance to the on-site user 108 in-person. This allows for a broadening of the network of sources that can work together to create a higher standard of care for patients, which may increase the level of sophistication the on-site user 108 is able to perform a medical procedure wit. Access to this broad network may, for example, decrease procedure times, increase quality of procedure results, and increase overall patient comfort.

In some embodiments, the local electronic device 104 and the remote electronic device 106 are smartphones. This allows for both devices to be readily available, for users of the devices to be largely familiar with several main controls of the device, and for both devices to be easily portable, which may add to the ease of establishing a system 100. In the embodiments where both electronic devices are smartphones, both devices may be video-enabled and be able to call the other device to create a remote electronic connection, which may add to the ease of operation of an application, website, or other such interface that allows the on-site user 108 and the operator of the remote electronic device 106 to communicate.

In other embodiments, one or more of the local electronic device 104 and the remote electronic device 106 are tablets, computers, or other pre-existing devices capable of electronic communication. In some embodiments, the local electronic device 104 is a video-enabled electronic device capable of videoing the medical procedure being performed on the patient 102 and transmitting that video to the remote electronic device 106. The remote electronic device 106 may be capable of videoing and sending such video to the local electronic device 104. In embodiments where the remote electronic device 106 is a video-enabled device, the user of the remote electronic device 106 may video, for example, themselves, other people, or another probe performing some kind of action or conveying some kind of information and send that video to the on-site user 108 to help the on-site user 108 complete a medical procedure. This video may be sent in real-time for immediate patient assistance or may be recorded and sent at a later time for later patient assistance or for later teaching and/or learning opportunities.

In some embodiments, one or more of the local electronic device 104 and the remote electronic device 106 are custom electronic devices. Such a custom electronic device may, for example, be optimized or created solely for the performing of actions useful in using or controlling the medical device and for creating the system 100 to allow a remote source to provide feedback to a local user of the probe 120.

The local electronic device 104 and the remote electronic device 106 may communicate via a remote interface. This interface may be shared such that the local electronic device 104 and the remote electronic device 106 display the same information simultaneously, or the interface may allow a user(s) of either device to customize what information is displayed. The interface may be the interface of an application or program that is downloaded on or otherwise connected to and accessed by both devices, or the interface may be a website that is accessed by both devices.

In some embodiments, the local electronic device 104 and the remote electronic device 106 can share an application that allows the two electronic devices to communicate via a shared application interface. In some embodiments, the application can be an individual program or application that is dedicated to a given function or purpose.

The remote user(s) of the remote electronic device 106 may optionally provide feedback to the on-site user 108 via this application. This feedback may include live or recorded audio feedback, or may include drawings, notifications, graphic overlays to the shared video streams, or other forms of written feedback.

For example, the local electronic device 104 may be used to video the medical procedure being performed by the on-site user 108 and transmit that video to the remote electronic device 106 though this application. One or more remote user(s) can view the transmitted video on the remote electronic device 106 also through this application. Additionally, the output of the probe 120, such as an ultrasonic scan image, can also be viewed on both electronic devices through the interface of this application. Both the on-site user 108 and any remote user of the remote electronic device 106 may also be able to control aspects of or outputs of the probe 120 though this application, including, for example, capturing and recording an image or video of the current ultrasound scan, and/or changing a setting of the probe 120 including brightness, depth, gain, presets, imaging modes including freeze/B-mode imaging and doppler/M-mode imaging, time gain compensation (TGC), needle visualization, thermal and mechanical indexes (Ti/Mi), DICOM destination, PACS settings, and any other image and/or video acquisition configuration settings.

In some embodiments, the local electronic device 104 and the remote electronic device 106 may communicate or otherwise share information over the internet or a local network. A website may provide a way for the local electronic device 104 and the remote electronic device 106 to communicate, share information, and/or control the probe 120.

In some embodiments, the probe 120 may broadcast directly to a monitor or other screen. Thus, instead of a system in which the local electronic device 104 conveys information from the probe 120 via an app to another user(s), the local electronic device 104 may be omitted from the system or may exist only for the limited purpose of videoing the medical procedure. Optionally, the local electronic device 104 can also provide a communications interface, such as a microphone and/or speaker, that can allow the on-site user 108 to communicate with the remote user of remote electronic device 106. Further optionally, the system can be configured such that the local electronic device 104 does not provide a means for the on-site user 108 to control the probe 120.

For example, in some embodiments, the on-site user 108 may only have limited control over the probe 120, such as the ability to physically move the probe 120 and turn the probe 120 on or off. The remote user of the remote electronic device 106 may have full control of the probe 120, such as the ability to change all possible settings of the probe 120 and the ability to record ultrasonic scan images or ultrasonic scan video.

Moreover, as noted herein, although the medical device of the system is shown as a probe 120, according to some embodiments, the medical device can also be any of a variety of other medical devices, whether handheld or not, and whether electronic or not. The systems and methods disclosed herein can advantageously be used to provide or improve communication and knowledge sharing between a group of individuals, including leveraging certain artificial intelligence functions that can enhance the performance of a medical procedure or aid in educating other on the medical procedure.

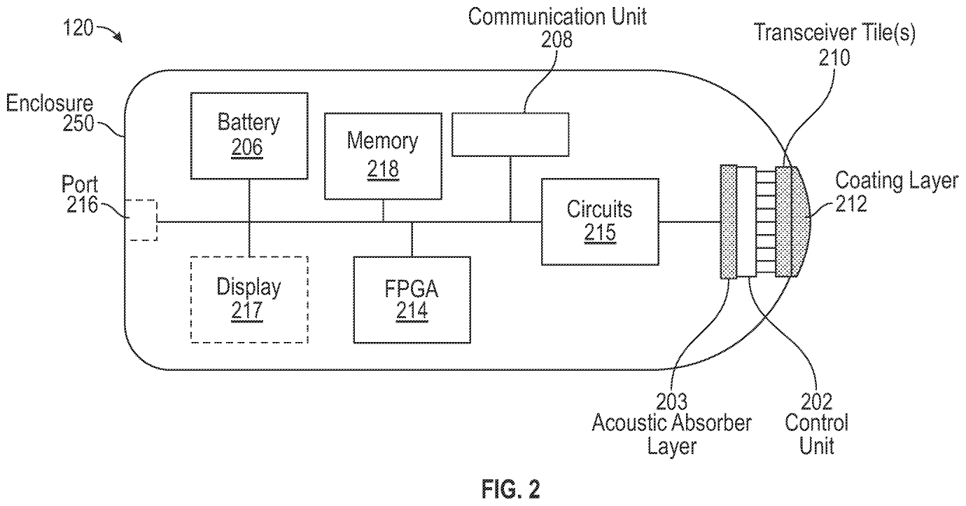

FIG. 2 shows a schematic diagram of the probe 120, according to some embodiments. In embodiments, the probe 120 may be an ultrasonic imaging device.

As depicted in FIG. 2, the probe 120 may include transceiver tile(s) 210 for transmitting and receiving pressure waves. A coating layer 212 may operate as a lens for steering the propagation direction of and/or focusing the pressure waves and may also function as an impedance interface between the transceiver tile 210 and the skin of the patient 102.

The probe 120 may further include a control unit 202, such as an application specific integrated circuit or ASIC chip (or, shortly ASIC), for controlling the transceiver tile(s) 210 and coupled to the transceiver tile 210 by bumps. Field Programmable Gate Arrays (FPGAs) 214 may control the components of the probe 120. Circuit(s) 215, such as an Analogue Front End (AFE), may exist for processing/conditioning signals.

An acoustic absorber layer 203 may absorb waves that are generated by the transceiver tiles 210 and propagate toward the circuit 215.

The probe 120 may further contain a communication unit 208 for communicating data with an external device, such as the local electronic device 104, the remote electronic device 106, and/or other devices, as discussed herein, through one or more ports 216. The probe 120 may also comprise a speaker, microphone, and/or other equipment for permitting communication and prompts to the on-site user 108.

A memory 218 may store data, and a battery 206 may provide electrical power to the components of the imager.

Optionally, the probe 120 may contain a display 217 for displaying images of, for example, ultrasonically scanned target organs.

In some embodiments, the local electronic device 104 may have a display/screen. In such embodiments, the display can advantageously be incorporated into the local electronic device 104 instead of or in addition to some portion of the probe 120. However, the system can be implemented to permit modular operation, viewing, and/or control of the probe and its operation. For example, the display generated from the probe 120 can be separate from the probe 120 itself and optionally, even separate from a device or controller that the on-site user 108 can use to control one or more functions or features of the probe 120.

In some embodiments, the probe 120 may receive electrical power from the local electronic device 104 through one of the ports 216. In such a case, the probe 120 may not include the battery 206. It is noted that one or more of the components of the probe 120 may be combined into one integral electrical element. Likewise, each component of the probe 120 may be implemented in one or more electrical elements.

In some embodiments, the user may apply gel on the skin of the patient 102 before the skin makes a direct contact with the coating layer 212 so that the impedance matching at the interface between the coating layer 212 and the skin of the patient 102 may be improved. In embodiments, the transceiver tiles 210 may be mounted on a substrate and may be attached to an acoustic absorber layer. This layer absorbs any ultrasonic signals that are emitted in the reverse direction, which may otherwise be reflected and interfere with the quality of the image.

As discussed below, the coating layer 212 may be only a flat matching layer just to maximize transmission of acoustic signals from the transducer to the body and vice versa. In embodiments, the thickness of the coating layer 212 may be a quarter wavelength of the pressure wave generated by the transceiver tile(s) 210. Beam focus in the elevation direction, which is along the direction of the length of the column can be electronically implemented in control unit 202. Even then, the lens may be designed with a focus in some cases. The probe 120 may use the reflected signal to create an image of an organ of the patient 102 and results may be displayed on a screen in a variety of format, such as graphs, plots, and statistics shown with or without the images of the organ.

In some embodiments, the control unit 202, such as an ASIC, may be assembled as one unit together with the transceiver tiles. In other embodiments, the control unit 202 may be located outside the probe 120 and electrically coupled to the transceiver tile 210 via a cable. In some embodiments, the probe 120 may include a housing or enclosure 250 that encloses the components of the probe 120 and a heat dissipation mechanism for dissipating heat energy generated by the components.

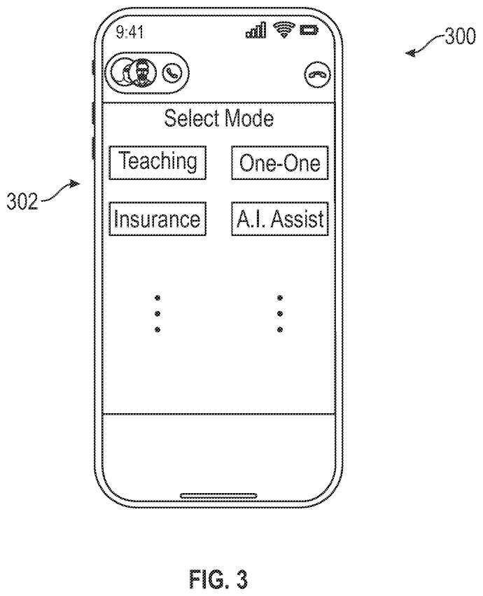

FIG. 3 shows an interface screen 300 of an application displaying mode option(s) 302, according to some embodiments. Each of the mode options 302 may correspond to a different mode of an application used to facilitate communication between the on-site user 108 and the remote user of remote electronic device 106, who is providing feedback to the on-site user 108.

For example, one available mode may be a teaching mode, where the remote user of the remote electronic device 106 is able to guide the on-site user 108 through a medical procedure being performed with the probe 120. This mode may include pre-determined teaching elements such as step-by-step guides, videos, or images that the on-site user 108 can access and review, and which the remote user can use, show, or reference to help the on-site user 108 in completing a medical procedure.

Another mode option 302 may include a one-one mode, where the on-site user 108 can call a remote user for help, and the remote user can provide feedback to the on-site user 108. For example, the remote user can create or provide various remotely generated graphic overlays that the remote user can superimpose onto the video of the procedure as it is being performed and transmitted from the local electronic device 104 to the remote electronic device 106, auditory feedback, video captured by the remote electronic device 106 and transmitted back to the local electronic device 104, or other forms of feedback. Controls may be available on the display screen to allow the experienced user to change the size, direction, or other attributes of the overlay.

Another mode option 302 may include an insurance mode, which helps the on-site user 108 in completing all the required steps of the medical procedure being performed with the use of probe 120. The insurance mode may, for example, advise the on-site user 108 of all of the required scans that are necessary in order to complete an ultrasonic imaging procedure in order to properly complete the procedure for insurance and/or billing purposes. Thus, the on-site user 108 may benefit from the guidance of another person or artificial intelligence so that a medical procedure can be documented appropriately for insurance and/or billing purposes.

Another mode option 302 may include an artificial intelligence (AI) assist mode, similar to a teaching or one-one mode, where instead of involving a remote skilled device user, the application guides the on-site user 108 on the use of the probe 120 by way of an artificial intelligence program.

Other mode options 302 may additionally be available to help the on-site user 108, and the on-site user 108 may switch between or combine different modes while using the application.

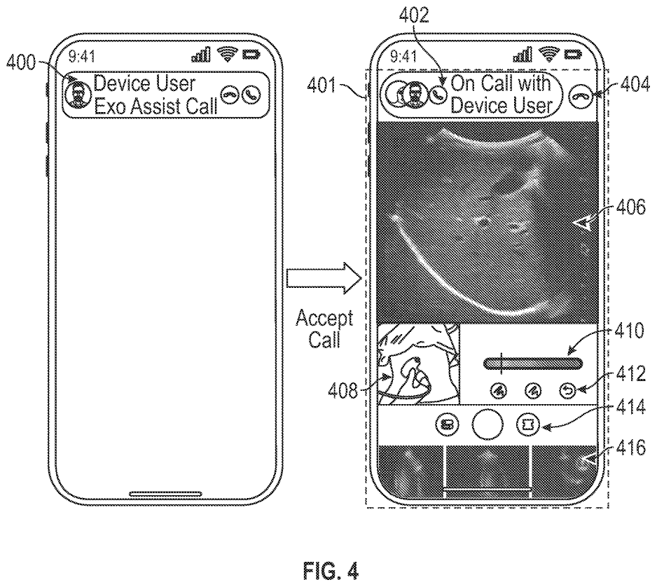

FIG. 4 shows two screens that may be used or presented when a remote user is receiving a call from the on-site user 108. On the left, a first screen provides an example call notification 400 that someone, such as an experienced user, may receive when the on-site user 108 calls them for help. On the right, FIG. 4 subsequently shows what a display screen 401 may look like on their remote electronic device 106 after accepting the call, according to some embodiments.

The display screen 401 may include a call information section 402 that may include information including the name of the caller/the on-site user 108, a profile picture of the caller/the on-site user 108, or other identifying information about the current call. The display screen 401 may further include icons such as an end call button 404, to be selected to end the call. In some embodiments where remote electronic device 106 is touchscreen enabled, buttons on the display screen 401, such as end call button 404, may be selected by tapping on the display screen 401.

The display screen 401 may further comprise a display of an ultrasound image 406 and/or a video feed 408. The display screen 401 may additionally comprise various selection options including a color selection bar 410, draw options 412, and image options 414. These various selection options may be used by the experienced user to provide feedback to the on-site user 108.

In some embodiments where the probe 120 is an ultrasonic imaging device, the display screen 401 may further show, for example, a list of captured images 416 of ultrasonic scans taken by the probe 120. In embodiments where the probe 120 is an ultrasonic imaging device, the ultrasound image 406 may be a live video feed of what is being scanned by the probe 120.

The video feed 408 may be a live video feed of the medical procedure being performed by the on-site user 108 on the patient 102 with the use of the probe 120. The color selection bar 410 may allow the experienced user to select a specific color with which to draw over the ultrasound image 406 to indicate something to the on-site user 108. The screen may also present draw options 412 and/or image options 414 that may be used by the remote user.

The draw options 412 may include selections such as a highlight marker or a solid color marker that the experienced user can select to use to draw with. The draw options 412 may further include selection buttons for an “undo” option that erases the last item drawn, or other similar quick tasks related to drawing feedback on the ultrasound image 406.

The image options 414 may include a selection button for taking a captured image 416 of what is being scanned and shown in the ultrasound image 406. These captured images 416 may be shown somewhere on the display screen 401.

Further, in some embodiments, an experienced user may be able to “toggle” between which video feed, of the ultrasound image 406 and the video feed 408, appear larger on the display screen 401 by, for example, tapping on the feed currently displayed as smaller to toggle it to the larger view, or by selecting a toggle button located, for example, within the image options 414.

In further embodiments of the disclosure, the experienced user may be able to select options displayed on the display screen 401 that allow the experienced user to alter aspects of the ultrasonic image. This may include, for example, changing aspects of the ultrasound image 406 by altering a setting of the probe 120 including brightness, depth, gain, presets, imaging modes including freeze/B-mode imaging and doppler/M-mode imaging, time gain compensation (TGC), needle visualization, thermal and mechanical indexes (Ti/Mi), DICOM destination, PACS settings, and any other image and/or video acquisition configuration settings.

The experienced user, in some embodiments of the disclosure, can therefore control aspects of the probe 120 that result in changes to the ultrasound image 406. This control may be possible, for example, through a wireless connection between the remote electronic device 106 and the probe 120. For example, the experienced user can remotely control or adjust features and/or settings such as mode selection, measurements (such as volume or velocity), calculations, adjustments to presets, brightness, scan settings (such as gain, scan depth, size/zoom, color, focus, or position), image and video capture, image freeze, annotation and capture tools, power settings, software settings, tool selection, share and data transfer settings and controls, and/or various other features and controls as may be implemented for a given medical device.

In accordance with some embodiments, the on-site user 108 can similarly have control over aspects of the ultrasonic image 406 via controls located on a similar display screen of the local electronic device 104 and the probe 120 itself.

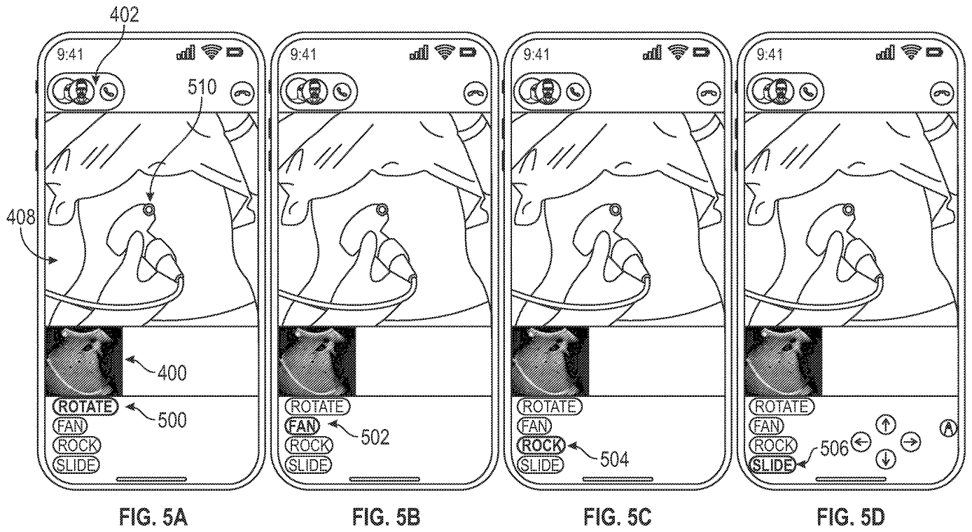

FIGS. 5A-5D show an example display screen, according to some embodiments including movement selection buttons 500, 502, 504, and 506. The example display screen of FIG. 5 further includes the video feed 408 and the ultrasound image 406.

As explained in the description of FIG. 4, the video feed 408 and the ultrasound image 406 may be toggled between which appears in a larger format on the screen. Further, according to some embodiments, an experienced user may select one of several preset movement selections, for example, as shown in FIGS. 5A-5D as the movement selection buttons 500 (rotate), 502 (fan), 504 (rock), and 506 (slide), discussed further below, which can bring up tools that the experienced user can utilize for providing feedback to the on-site user 108 about how to operate the probe 120 while completing a specific motion. These specific motions may correspond to certain “best practice” motions for operating the probe 120, where some motions may be more successful in capturing a successful scan image during different procedures.

Further, the probe 120 may include a probe indicator 510. This indicator 510, as a physical marker or indicia on the probe 120, may be visible both to the on-site user 108 and the experienced user viewing the live video 408. The indicator 510 can correspond to a physical reference point or feature of the probe 120 and may be useful in illustrating or determining in which direction the probe 120 is currently being held and for aligning the probe according to the feedback given. Accordingly, the on-site user 108 can adjust the probe position based on the illustrated location or alignment of the indicator 510.

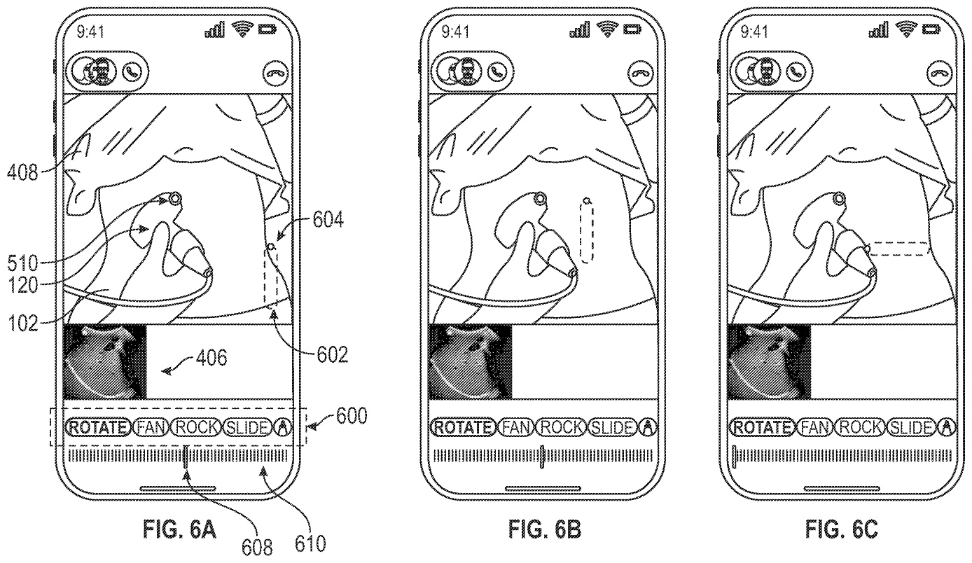

FIGS. 6A-6D show an example device display screen, according to some embodiments, where the experienced user has selected a specific “rotate” movement from the overlay selection section 600. By selecting an overlay type, the experienced user is able to utilize a graphic overlay 602 that corresponds to the chosen movement in providing feedback to the on-site user 108. In some embodiments, the selected motion, as, for example, depicted in FIGS. 6A-6D, may include a “rotate” motion. However, as discussed further herein, other motions can be selected and modeled by the remote user for replication by the on-site user.

In some embodiments of the disclosure, the device display screen may show, for example, a live video feed of the procedure that includes showing the probe 120 as being used on the patient 102. Further, the probe 120 may include the probe indicator 510 that assists in the device user and the experienced user understanding, for example, in which direction the probe is being held.

The graphic overlay 602 may also include an overlay indicator 604 that may be used to help the device user and the experienced user understand, for example, where and/or how the on-site user should move the probe 120 first and then begin the movement as illustrated by the graphic overlay. Thus, the graphic overlay 602 can model the position, movement, and/or articulation of the movement that the on-site user can then replicate using the physical probe 120. Additional controls may be available on the display screen to allow the experienced user to change the size, direction, or other attributes of the overlay.

The device display screen may further include video feed showing the ultrasound image 406 currently being generated by the probe 120, allowing the experienced user to see, for example, how the on-site user 108 moving the probe 120 affects the ultrasound image 406.

Additionally, according to some embodiments, the experienced user may be able to control the orientation of the overlay 602 by way of a slider 608 located on the display screen in order to illustrate a “rotate” motion. The experienced user may, for example, be able to click and drag the slider 608 along the slider bar 610, and in so doing move the orientation of the overlay 602 to visually illustrate a desired rotation motion of the probe 120, based on the location of the slider 608 along the slider bar 610.

For example, the slider 608 set to the far left of the slider bar 610 may result in the overlay 602 being horizontal, with the overlay indicator 604 located at the far left of the overlay 602; the slider 608 set to the middle of the slider bar 610 may result in the overlay 602 being vertical, with the overlay indicator 604 located at the top of the overlay 605, with the “top” direction being nearest to the top of the display screen. Any position of the slider 608 between the far left and the center of slider bar 610 would result in the overlay 602 having some angular offset from horizontal or vertical, and a similar orientation process would result from positioning the slider 608 along the right side of the slider bar 610. Accordingly, a rotation of the overlay 602 can be visually presented to the on-site user so that the on-site user can model the illustration using the physical probe 120 and thereby enhance the performance of the medical procedure.

In some embodiments, the midpoint of the slider bar 610 controls the overlay 605 to have 0 degrees of rotation. When the remote user slides the slider bar to the right, it orients the overlay 605 accordingly, and guides the on-site user of the probe to rotate the probe clockwise. When the remote user slides the slider bar to the left, it guides the user of the probe to rotate the probe counterclockwise. The slider bar may be scaled to control the amount of rotation required. In some embodiments, haptic feedback may be used to alert the on-site user of the probe when the probe is accurately positioned.

Further controls may additionally be available to enable the experienced user to change the size and precise location of the overlay as displayed on the live video feed of the procedure. In some embodiments, augmented reality features may allow the overlay to be superimposed over the image or video feed such that if the position of the imaging device moves such that the video moves or the perspective changes, the overlay should adapt to remain in the correct location relative to the patient.

Accordingly, in some embodiments, the experienced user can control the overlay 602 via the remote electronic device 106. In real-time, the on-site user 108 can see the overlay 602 appear over the video feed 408 shown on the screen of local electronic device 104. The on-site user 108 can then move the probe 120 to correspond to the feedback being conveyed by the overlay.

Additionally, in some embodiments, where the experienced user is operating the application on a touchscreen device, the experienced user could click and drag the overlay 602 to position it in a desired location over the video feed, zoom in to the video feed 408, etc.

Further, in some embodiments, notifications may appear on the screen of the local electronic device 104 so the on-site user 108 can be informed of the instructions provided by the experienced user. These notifications may include text prompts, including instructions on how or where to move the probe or adjust a setting, symbols including arrows or other icons, or other visual or auditory prompts. These notifications may further include information to convey that someone on the call changed a setting or made or suggested any changes to the use of the probe 120.

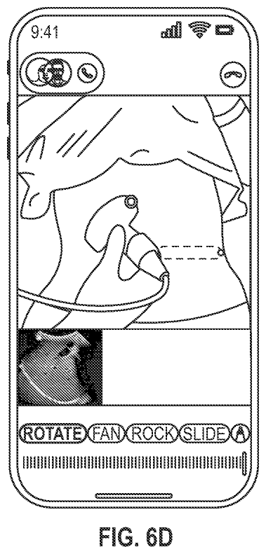

FIGS. 7A-7F show another example device display screen, according to various embodiments, where the experienced user can select a “fan” movement from the overlay selection section 600.

In some embodiments, this selected motion may include a “fan” motion, where the on-site user 108 is guided to move the probe in a fanning motion, including holding the distal end of the probe 120 steady against the skin of the patient 102, while moving the proximal, or hand-held end of the probe 120 back and forth in a semi-circular arc shape.

To prompt the on-site user 108 to make this “fan” motion with the probe 120, the experienced user may select the graphic overlay 702 to add over the video feed 408. This overlay 702 may, for example, be generally shaped according to how the probe 120 is shaped, to show the on-site user 108 where they should place the probe 120 relative to the patient 102. Therefore, the on-site user 108 can match the position, motion, and articulation of the probe 120 to the guided position of the overlay 702.

Further, to guide the on-site user 108 through the “fan” motion, the experienced user may sequentially select and show a series of overlays 704, 706, 708, 710, to show the on-site user 108 how the angle of the probe 120 should change during this motion. Alternatively, the application may be able to display these overlays 704, 706, 708, 710 in a preset sequence, or alternatively, the application may be able to show the “fan” motion in an overlay as a video animation.

In some embodiments, the experienced user may be able to orient the location of the overlay 702 using the slider 608 and slider bar 610, where sliding the slider 608 along the slider bar 610 corresponds to changing the displayed “fan” angle of the overlay 702. The slider bar 610 may be controlled by the remote user to precisely control the positioning of the overlay 702 to provide quantitative feedback to the on-site user 108 regarding the recommended probe positioning. Additional controls may be available on the display screen to allow the experienced user to change the size, direction, or other attributes of the overlay. Additionally, or alternatively, the experienced user may be able to click and drag the location of the overlay 702 to change its size, direction, or other attributes.

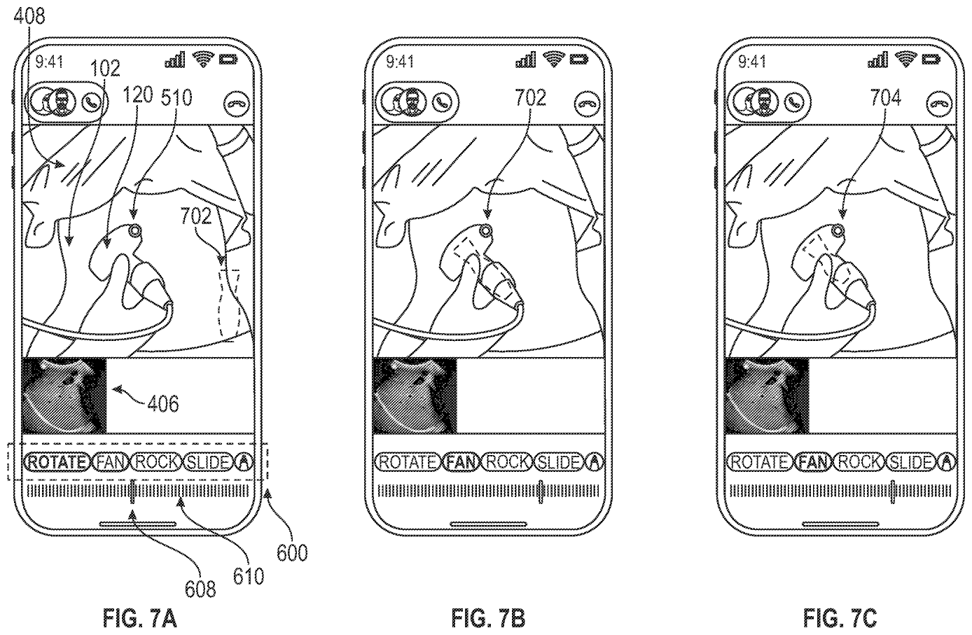

FIGS. 8A-8F show another example device display screen according to various embodiments where the experienced user has selected a “rock” movement from the overlay selection section 600.

In some embodiments, this “rock” motion may include a “rock” motion, where the on-site user 108 is guided to move or pivot the probe 120 in a rocking motion.

For example, the on-site user 108 can hold the probe 120 against the skin of the patient 102 and moving the handle portion of the probe 120 back and forth in a generally left to right direction. The terms “left” and “right” are taken to mean to the left of and to the right of a center axis of the probe, lengthwise along its longest direction. The slider bar 610 may be controlled by the remote user to precisely control the positioning of the overlay 802 to provide quantitative feedback to the on-site user 108 regarding the recommended probe positioning.

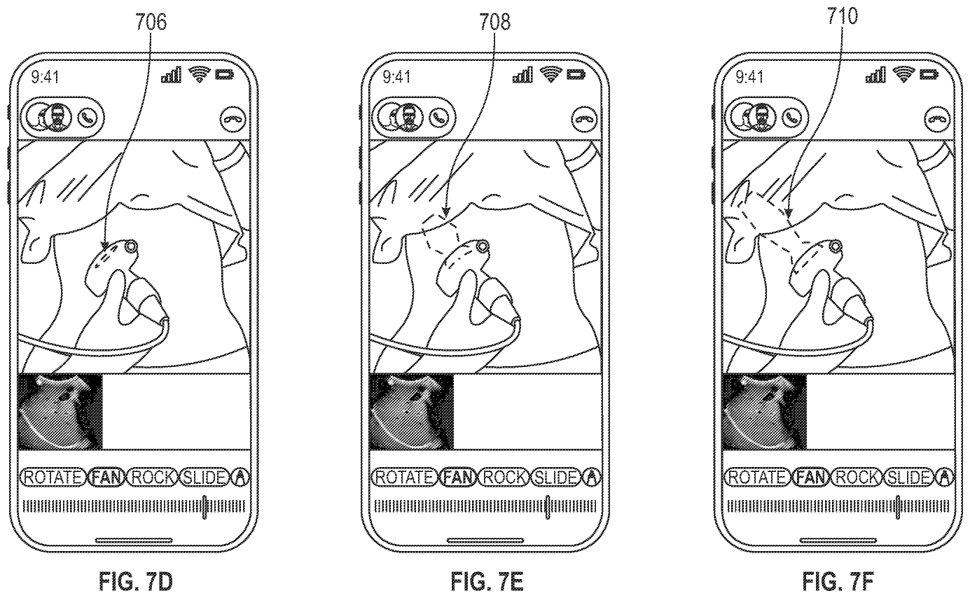

FIGS. 9A-9D show another example device display screen according to various embodiments where the experienced user has selected a “slide” movement from the overlay selection section 600.

In some embodiments, this “slide” motion may include a “slide” motion, where the on-site user 108 is guided to move the probe 120 in a sliding motion, including translating the probe 120 back and forth along a line over the skin of the patient 102.

In some embodiments of the disclosure, the experienced user may select a graphic overlay 902 to indicate that the on-site user 108 should move the probe 120 in this “slide” motion. The overlay 902 may include an overlay indicator 904, which can help the experienced user and the device user to orient where the slide motion should begin on the patient 102.

As noted above with respect to other potential features, the experienced user may use the slider 608 along the slider bar 610 along with other available controls to orient the overlay 902. The slider bar 610 may be controlled by the remote user to precisely control the positioning of the overlay 902 to provide quantitative feedback to the on-site user 108 regarding the recommended probe positioning. Alternatively, the experienced user may click and drag the overlay 902 to change its orientation, size, or other attributes as displayed on the video feed 408.

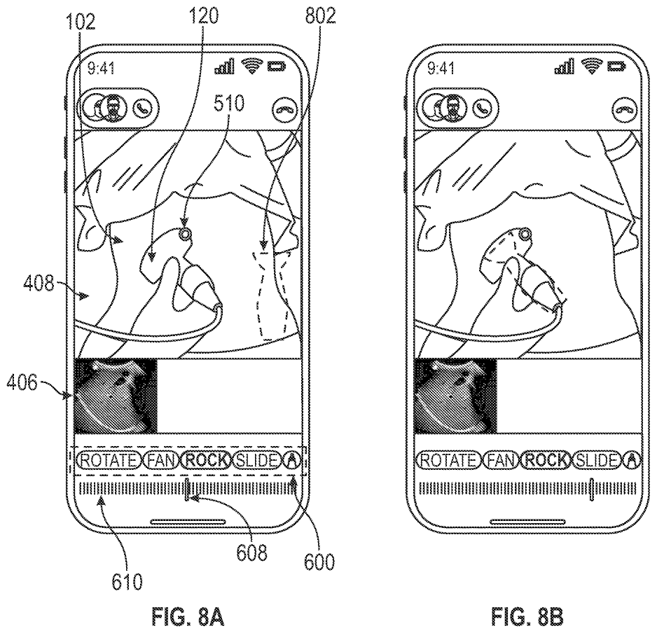

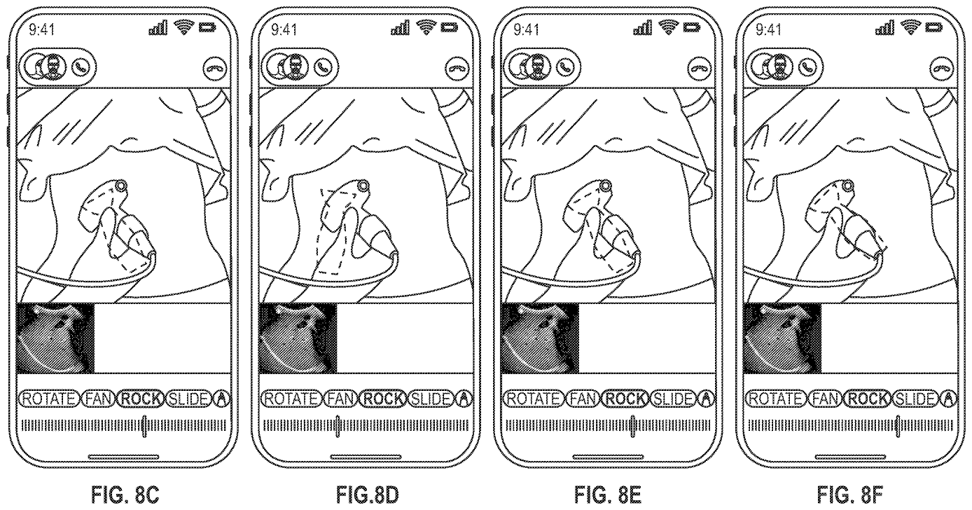

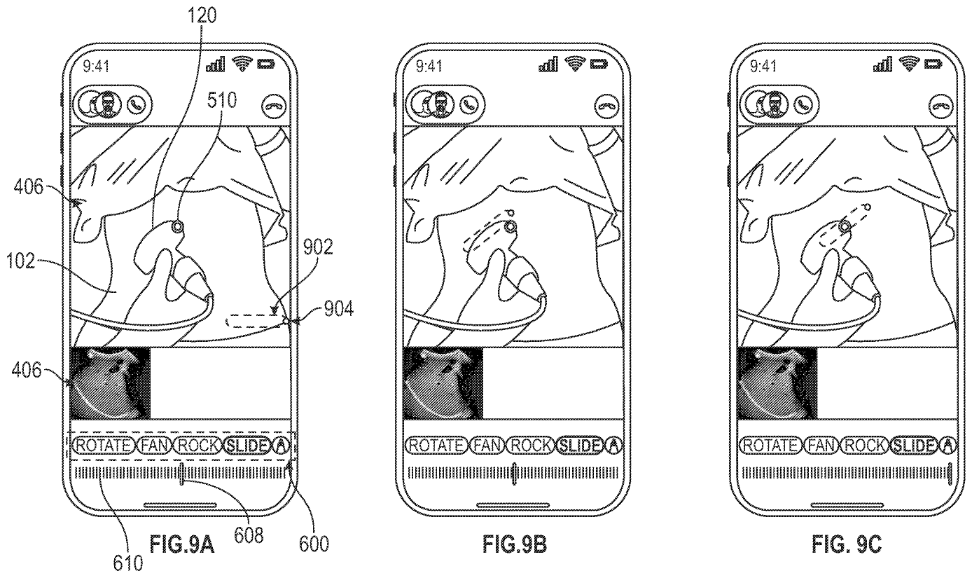

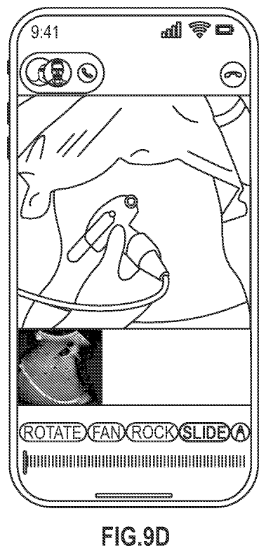

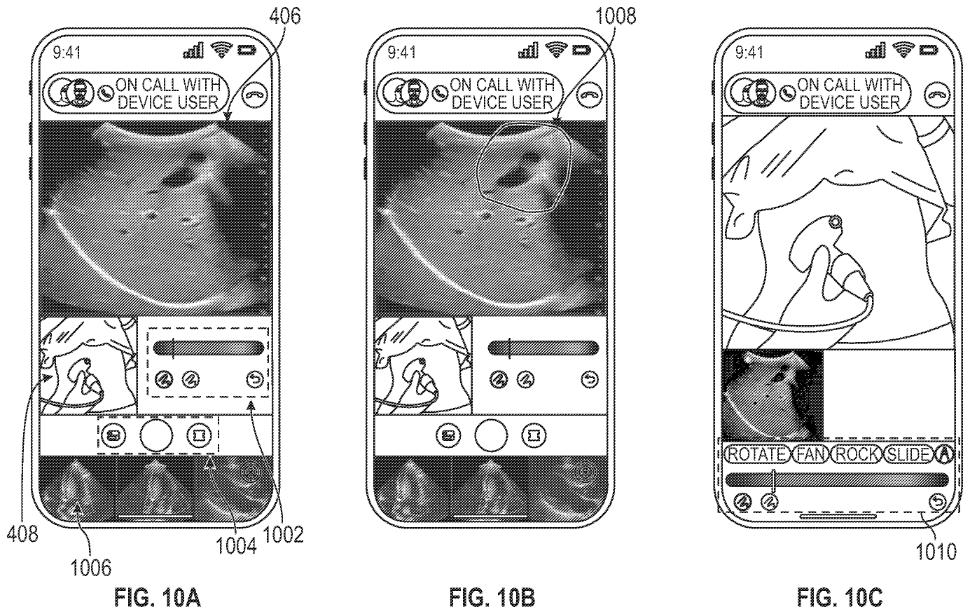

FIGS. 10A-10E show an example device display screen according to various embodiments where the experienced user is able to select various drawing options from the drawing controls 1002 and is able to hand draw over either the ultrasound image 406 or the video feed 408 to communicate feedback to the on-site user 108. This drawn feedback may be conveyed through various free-form markings, such as depicted, for example, by the drawn overlays 1008 and 1016.

For example, the feedback or markings may include circles, X's, or other free-form drawings including lines, ovals, arrows, curves, or various other possible sketches. Preset shapes may additionally be available for the experienced user to select from and position on the video feed. The available drawing options may further include, for example, a color bar to select what color to draw with, options for a highlighter mode (e.g., translucent markings) or a pen mode (e.g., opaque markings), an ability to change the size or thickness of the marking being drawn, the ability to erase markings, “undo” the last drawn marking, or other drawing controls.

The example device display screen, according to various embodiments, may further comprise a control section 1004 comprising, for example, various buttons relating to common actions, such as a button to capture an image of the current ultrasound scan being generated, a button to toggle between the video feed 408 and the ultrasound image 406, a button to view drawing controls, a button to view the overlay options, a button to connect/disconnect phone calls, a button to turn on/off the camera, a button to add additional users to the call, a microphone mute/unmute button, zoom options, movement controls, or other settings.

In various example device display screens, captured images 1018 may be recorded and displayed somewhere on the screen, or may be able to be viewed upon selecting a corresponding button. In other example device display screens, the controls may be arranged in different locations or groupings, such as in overlay controls 1010. The various example device display screens shown are representative of various contemplated designs, but do not comprise all possible variations of the application's display.

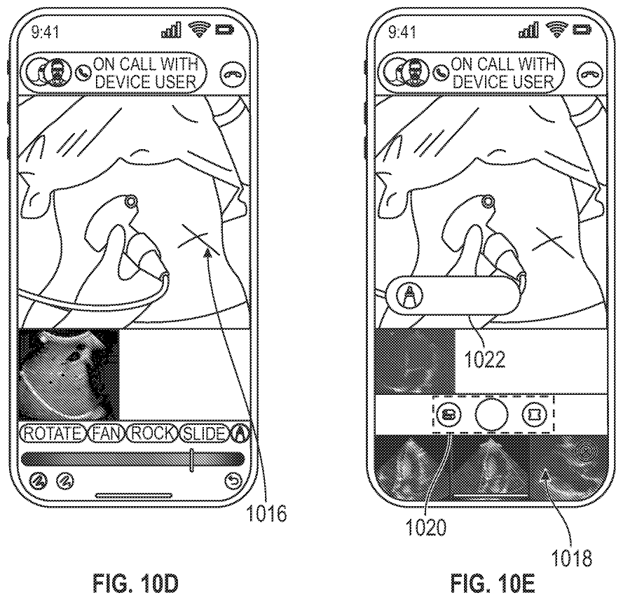

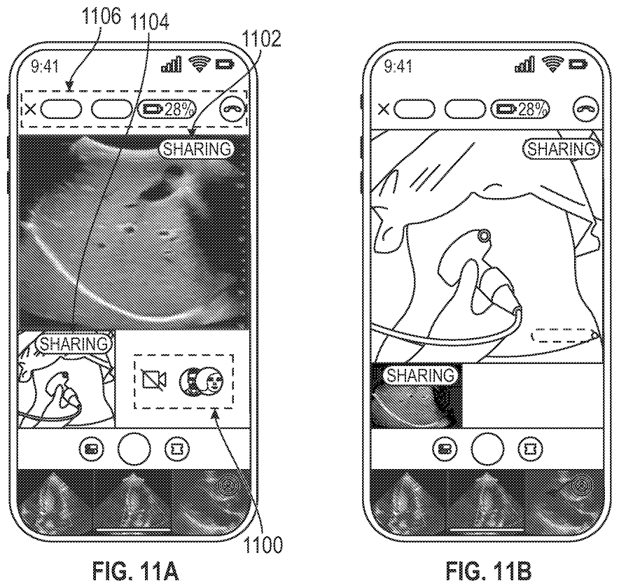

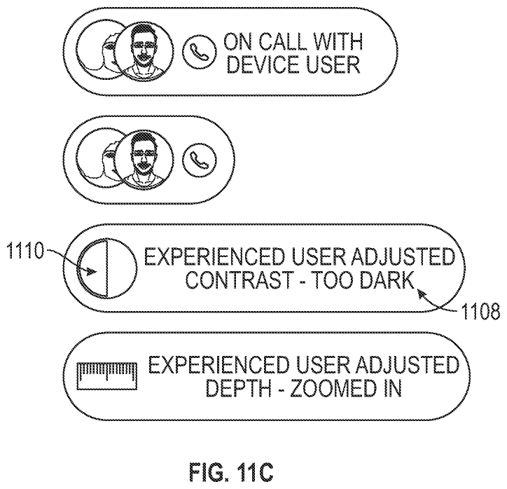

FIG. 11A shows an example device display screen according to various embodiments illustrating various additional notifications that may be present on the display screen. For example, icons 1100 may be present to identify various aspects of the call such as identify who is participating in the call, or provide further controls related to the call, such as provide the ability to mute the call, pause the video feed, or other related controls. In embodiments of the disclosure where the local electronic device 104 is being used to capture and transmit the video feed 408, certain controls relating to the video feed may only be available to the user of the local electronic device 104. These controls may include, for example, the ability to start, stop, or pause the video feed 408 or adjust various settings relating to the probe 120. Alternatively, the experienced user can also access the same video controls the device user is able to access.

Additional icons may be present on the device display screen, including within information sections 1102, 1104, and 1106. These sections may include text notifications, such as whether or not the displayed screen is being shared with another connected device, or other previously discussed notifications.

FIG. 11B shows an alternative view to the device display screen shown in FIG. 11A, where here the user of the device has toggled the video feed 408 to a larger view. In other embodiments, the video feed 408 and ultrasound image 406 may be shown in equally large views, one may be shown in a full-screen mode, or other possible variations on the size of the display may be possible.

FIG. 11C shows possible notifications, according to embodiments, where a notification may include notification text 1108 and/or a notification graphic 1110. In various embodiments, notifications may include various amounts of text, various numbers of graphics, including no graphics, and may be accompanied by an audio component such as a notification sound or a haptic feedback component including, for example, a vibration felt in the electronic device being used to operate the application or a vibration felt in the probe 120.

In some embodiments, the experienced user may have a second probe similar to the probe 120, wherein the experienced user's probe contains an accelerometer than can communicate with an accelerometer located within the probe 120, such that the experienced user can manipulate the second probe to communicate with the probe 120. By changing, for example, the angle at which the experienced use holds the second probe, the on-site user 108 may feel haptic feedback that guides their own probe 120 into the same positioning as the second probe. Similarly, such haptic feedback may be provided by an AI program during the medical procedure.

In some embodiments, the on-site user 108 may be prompted to complete a series of actions that are necessary in order to fulfill the requirements of insurance billing. Through the interface of the local electronic device 104, for example, the on-site user 108 may be given a set of directions. These directions may include what scans are necessary for the user 108 to complete in order to complete the procedure. These directions may further include how to capture each required scan, such as best-practices for obtaining each required image. These directions may be provided to the user 108 though a list, a series of images, or a series of videos.

In some embodiments, the experienced remote user may prompt the more novice on-site user to capture the minimum required views or scans required for billing and insurance purposes. In some embodiments, an AI program may prompt the on-site user to capture the minimum required views or scans required for billing and insurance purposes. The AI program may additionally prompt the on-site user to complete the required paperwork and use the required billing codes. Based on selected presets, the on-site user can be prompted to add relevant values to quickly complete associated worksheets, including indications, views, interpretations, and findings.

In some embodiments, with the help of AI, the on-site user may be shown what a normal or healthy ultrasound scan looks like to help the on-site user understand any abnormalities they are viewing in their ultrasound imaging feed. This may be accomplished by showing prompts on the imaging screen displaying visual cues to indicate “abnormalities” that they are viewing. In some embodiments, the on-user may be prompted to see what a normal or healthy scan looks like to help confirm their assessment. AI may also be used to provide customized real-time guidance to the on-site user by showing relevant training videos or images to watch in real-time or save for later viewing.

In some embodiments, the user 108 is guided by a more experienced practitioner through all the steps of a medical procedure that must be completed in order to bill a patient's insurance. The more experienced practitioner may communicate feedback to the user 108 through the remote electronic device 106, which the user 108 may be able to view on the local electronic device 104.

In some embodiments, an AI program may exist within the interface to guide the user 108 through successful completion of a medical procedure in accordance with what must be completed in order to bill a patient's insurance. For ultrasonic scan procedures, the AI program may be able to receive a proposed image from the on-site user 108 and compare that image to a known or preset required image for a set procedure. The AI program may be able to then determine whether the proposed image generated by the on-site user 108 is sufficiently similar to a known or preset required image of the set procedure.

This determination of whether the proposed image is sufficiently similar to a known required image may include determinations of a variety of image factors including image size, quality, or resolution, and/or the subject of the image including, for example, what organ is being scanned and in what orientation the image is generated. Image qualities and/or results that may also be considered include, for example, depth, gain, preset, measurement, findings, and interpretations.

The AI program may further be able to notify the on-site user 108, through the interface on the local electronic device 104, whether or not the proposed image was sufficient. If the proposed image was deemed sufficient to be submitted for insurance billing, the AI program may then prompt the on-site user 108 to move on to the next required image. If the proposed image was deemed insufficient, the AI program may help the on-site user 108 to adjust their scanning settings or technique in order to generate the scan required. This guidance may include any of the feedback previously described, including, for example, showing graphic overlays over a shared video that describe specific best-practice movements of the probe 120.

In some embodiments, the interface may link to documentation or paperwork that must be filled out and submitted with an insurance reimbursement request. The AI program may track which scans have been completed and may auto-populate information into the required forms, such as what procedure is being conducted, who conducted the procedure, and when and where the procedure took place. The interface may additionally aid the on-site user 108 in submitting completed insurance forms to various insurance providers.

### Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.
Clause 1. A method for providing remote guidance from a skilled resource to a local user during a medical procedure on a patient for enhancing the quality of the medical procedure performed by the local user, the method comprising: transmitting information, representative of a procedure being performed by the local user, to the skilled resource, wherein the local user performs the procedure using a medical device; permitting the skilled resource to transmit feedback to the local user; and conveying the feedback to the local user via a communication interface.
Clause 2. The method of Clause 1, wherein the permitting the skilled resource to transmit feedback to the local user comprises permitting the skilled resource to indicate at least one suggested position or movement of the medical device.
Clause 3. The method of Clause 2, wherein the at least one suggested position or movement comprises fanning the medical device, rocking the medical device, sliding the medical device, rotating the medical device, translating the medical device, or tilting the medical device relative to the patient.
Clause 4. The method of any of the preceding Clauses, wherein the transmitting information comprises sending a video feed to the skilled resource of the local user performing the medical procedure.
Clause 6. The method of Clause 4, wherein the video feed is a live video feed.
Clause 6. The method of any of the preceding Clauses, further comprising permitting the skilled resource to create an animation of the at least one suggested position or movement.
Clause 7. The method of any of the preceding Clauses, further comprising permitting the skilled resource to create an animation of the at least one suggested position or movement, wherein the animation is selected from a plurality of preset animations.
Clause 8. The method of Clause 6 or 7, further comprising displaying the animation to the local user.
Clause 9. The method of any one of Clauses 6-8, wherein the animation is overlaid onto the video feed.
Clause 10. The method of any one of Clauses 6-9, wherein the animation is overlaid onto the video feed in real time.
Clause 11. The method of any of the preceding Clauses, wherein the transmitting information comprises providing one or more images to the skilled resource.
Clause 12. The method of any of the preceding Clauses, wherein the permitting the skilled resource to transmit feedback comprises the skilled resource overlaying notation over the information transmitted to the skilled resource.
Clause 13. The method of any of the preceding Clauses, wherein the conveying the feedback comprises providing visual feedback to the local user using a first communication device.
Clause 14. The method of any of the preceding Clauses, wherein the conveying the feedback comprises transmitting an audio instruction to the local user.
Clause 15. The method of any of the preceding Clauses, wherein the conveying the feedback comprises providing a graphic overlay of an example routine, target area on the patient, position of the medical device, or movement of the medical device.
Clause 16. The method of any of the preceding Clauses, wherein the skilled resource comprises a skilled user of the medical device.
Clause 17. The method of any of the preceding Clauses, wherein the skilled resource comprises an artificial intelligence software.
Clause 18. The method of any of the preceding Clauses, wherein the communication interface comprises a display.
Clause 19. The method of any of the preceding Clauses, wherein the communication interface comprises a smartphone.
Clause 20. The method of any of the preceding Clauses, wherein the medical device comprises an imaging probe.
Clause 21. The method of any of the preceding Clauses, further comprising permitting the skilled resource to transmit an example routine to the local user for viewing.
Clause 22. The method of any of the preceding Clauses, wherein one or more steps of the method is performed by the local user using a first communication device.
Clause 23. The method of Clause 22, wherein the transmitting information is performed using the first communication device.
Clause 24. The method of Clause 22 or 23, wherein the conveying the feedback is performed using the first communication device.
Clause 25. The method of any one of Clauses 22-24, wherein the first communication device comprises a smartphone.
Clause 26. The method of any one of Clauses 22-25, wherein the first communication device comprises a tablet.
Clause 27. The method of any of the preceding Clauses, wherein one or more steps of the method is performed by the skilled resource using a second communication device.
Clause 28. The method of Clause 27, wherein the permitting the skilled resource to transmit feedback is performed using the second communication device.
Clause 29. The method of Clause 27 or 28, wherein the transmitting information is performed using the second communication device.
Clause 30. The method of Clause 29, wherein the transmitting information comprises displaying a video feed to the skilled resource.
Clause 31. The method of any one of Clauses 27-30, wherein the second communication device comprises a smartphone.
Clause 32. The method of any one of Clauses 27-31, wherein the second communication device comprises a tablet.
Clause 33. The method of any of the preceding Clauses, wherein the skilled resource is separate from and not carried by the medical device.
Clause 34. The method of any of the preceding Clauses, wherein the permitting the skilled resource to transmit feedback comprises permitting control of at least one attribute of the medical device by the skilled resource.
Clause 35. The method of Clause 34, wherein the permitting control comprises permitting the skilled resource to adjust an image taken by the medical device by adjusting a depth of focus, power, control the image features from the other side, screen, pause a video feed, align the image, etc.
Clause 36. The method of Clause 34 or 35, wherein the permitting control comprises controlling a setting or operation of the medical device.
Clause 37. The method of any one of Clauses 34-36, wherein the permitting control comprises capturing an image or video related to the medical procedure via the medical device.
Clause 38. The method of any one of Clauses 34-37, wherein the permitting the skilled resource to transmit feedback comprises permitting control of the at least one attribute of the medical device via a first communication device of the local user by the skilled resource.
Clause 39. The method of any of the preceding Clauses, wherein the permitting the skilled resource to transmit feedback to the local user comprises permitting the skilled resource to mark a virtual target on the patient or annotate the video feed with a symbol, line, or highlight tool.
Clause 40. A method for enhancing a device operator's use of a medical device via feedback from a remote source during a medical procedure, the method comprising: performing a medical procedure using the medical device; transmitting a video, via a shared application, of the medical procedure from a video-enabled device of the device operator to a remote device of the remote source, the shared application permitting sharing and viewing of the video; and permitting the remote source control of an operation or attribute of the medical device via the shared application.
Clause 41. The method of Clause 40, further comprising permitting the remote source to transmit feedback to the device operator.
Clause 42. The method of Clause 41, wherein the permitting the remote source to transmit feedback comprises the remote source overlaying notation over the video transmitted to the remote source.
Clause 43. The method of Clause 41 or 42, wherein the permitting the remote source to transmit feedback comprises permitting the remote source to mark a virtual target on a patient or annotate the video with a symbol, line, or highlight tool.
Clause 44. The method of any one of Clauses 41-43, wherein the permitting the remote source to transmit feedback comprises the remote source creating an animation of at least one suggested position or movement of the medical device.
Clause 45. The method of Clause 44, wherein the animation is selected from a plurality of preset animations.
Clause 46. The method of Clause 44 or 45, wherein the animation is overlaid onto the video feed.
Clause 47. The method of any one of Clauses 44-46, wherein the animation is overlaid onto the video feed in real time.
Clause 48. The method of any one of Clauses 41-47, further comprising conveying the feedback to the device operator via a communication interface.
Clause 49. The method of Clause 48, wherein the communication interface comprises a display.
Clause 50. The method of Clause 48 or 49, wherein the communication interface comprises a smartphone.
Clause 51. The method of any one of Clauses 48-50, wherein the conveying the feedback comprises providing visual feedback to the device operator.
Clause 52. The method of any one of Clauses 48-51, wherein the conveying the feedback comprises providing a graphic overlay of an example routine, target area on a patient, position of the medical device, or movement of the medical device.
Clause 53. The method of any of Clauses 40-52, further comprising permitting the remote source to transmit an example routine to the device operator for viewing.
Clause 54. The method of any of Clauses 40-53, wherein the transmitting a video is performed using a smartphone.
Clause 55. The method of Clause 54, wherein smartphone is operated by the device operator.
Clause 56. The method of Clause 41, wherein the remote source's device is a smartphone.
Clause 57. The method of Clause 56, wherein the permitting the remote source to transmit feedback is performed using the remote source's smartphone.
Clause 58. The method of any of Clauses 40-57, wherein the medical device is an imaging device.
Clause 59. The method of Clause 58, wherein the medical device is an ultrasonic imaging probe.
Clause 60. The method of Clause 59, wherein the permitting the remote source to control an operation or attribute of the medical device comprises permitting the remote source to adjust an image taken by the medical device by adjusting a depth of focus, power, control the image features from the other side, screen, pause the video feed, align the image, etc.
Clause 61. The method of Clause 59 or 60, wherein the permitting the remote source to control an operation or attribute of the medical device comprises capturing an image or video related to the medical procedure via the medical device.
Clause 62. The method of any one of Clauses 58-61, wherein the medical procedure is an imaging procedure.
Clause 63. The method of any of Clauses 40-62, wherein the permitting the remote source control comprises permitting the remote source to capture an image or video of the medical procedure via control of the medical device.
Clause 64. The method of any of Clauses 40-63, wherein the permitting the remote source control comprises permitting the remote source to change a mode of the medical device.
Clause 65. The method of any of Clauses 40-64, wherein the permitting the remote source control comprises, in response to a signal from the remote source, generating haptic feedback via the medical device to allow the remote source to communicate with the medical device operator.
Clause 66. The method of Clause 65, wherein the medical device contains an accelerometer, the remote source has a second medical device that contains an accelerometer, and the remote source can manipulate the second medical device such that when the medical device operator has oriented the medical device in the same way as the remote source, the medical device operator receives haptic feedback in their medical device.
Clause 67. A method of guiding an operator of a medical imaging device through completion of a medical procedure, the method comprising: receiving an indication that a proposed image submission has been acquired by the medical imaging device; comparing the proposed image submission against a submittal requirement to generate a submittal assessment representative of whether the submittal requirement has been met; and in response to the submittal assessment, notifying the operator whether to acquire an additional proposed image submission to meet the submittal requirement.
Clause 68. The method of Clause 67, wherein the receiving the indication comprises receiving the proposed image submission from the medical imaging device.
Clause 69. The method of Clause 68, wherein the comparing comprises comparing the proposed image submission against a database of images.
Clause 70. The method of Clause 68 or 69, wherein the comparing is performed using an artificial intelligence software.
Clause 71. The method of any of Clauses 67-70, wherein the submittal requirement identifies a required image necessary to complete an insurance reimbursement request, and wherein the comparing comprises verifying whether the proposed image submission is the required image of the submittal requirement.
Clause 72. The method of any of Clauses 67-71, wherein the submittal requirement identifies an image attribute necessary to complete an insurance reimbursement request, and wherein the comparing comprises verifying whether the proposed image submission satisfies the image attribute of the submittal requirement.
Clause 73. The method of any of Clauses 67-72, wherein the notifying comprises providing a visual indication to the operator via a communication interface that is in communication with the medical imaging device.
Clause 74. The method of any of Clauses 67-73, further comprising permitting the operator to submit a medical reimbursement request based on the proposed image submission.
Clause 75. The method of Clause 74, wherein the method is performed using an application software that permits the operator to create the medical reimbursement request when the submittal requirement is fulfilled.
Clause 76. The method of any of Clauses 67-75, wherein the medical imaging device is an ultrasonic probe.
Clause 77. The method of any of Clauses 67-76, wherein the guiding an operator of a medical imaging device is performed by a person skilled in use of the medical imaging device.
Clause 78. The method of any of Clauses 67-77, wherein the guiding an operator of a medical imaging device is performed by an artificial intelligence software.
Clause 79. The method of Clause 78, wherein the artificial intelligence compares the proposed image submission to a pre-programmed set of required scans to determine whether a scan being performed by the operator is sufficient to satisfy preprogrammed requirements.
Clause 80. A system for enhancing use of a medical device via feedback from a remote source during a medical procedure, the system comprising: a medical device for performing a medical procedure; a shared application software for communicating with the medical device; a camera-enabled first device configured to run the shared application software and permit visual capture of the medical procedure; a remote second device configured to run the shared application software and receive images or video of the medical procedure from the first device via the shared application, the second device being configured to permit the remote source to provide feedback based on performance of the medical procedure and to control an operation or attribute of the medical device via the shared application software for enhancing use of the device by a device operator.
Clause 81. The system of Clause 80, wherein the second device being configured to permit the remote source to provide feedback allows the remote source to provide visual feedback using the remote second device.
Clause 82. The system of Clause 81, wherein the visual feedback comprises a graphic overlay of an example routine, target area on a patient, position of the medical device, or movement of the medical device.
Clause 83. The system of Clause 81 or 82, wherein the visual feedback comprises an animation of at least one suggested position or movement of the medical device.
Clause 84. The system of Clause 83, wherein the animation is overlaid onto a video of the medical procedure.
Clause 85. The system of any of Clauses 80-84, wherein the first device is video enabled.
Clause 86. The system of any of Clauses 80-85, wherein the second device being configured to permit the remote source to provide feedback allows the remote source to provide auditory feedback.
Clause 87. The system of any of Clauses 80-86, wherein the first device is a smartphone.
Clause 88. The system of any of Clauses 80-87, wherein the first device is a tablet.
Clause 89. The system of any of Clauses 80-88, wherein the second device is a smartphone.
Clause 90. The system of any of Clauses 80-89, wherein the second device is a tablet.
Clause 91. The system of any of Clauses 80-90, wherein the medical device is an imaging device.
Clause 92. The system of Clause 91, wherein the medical device is an ultrasonic imaging probe.
Clause 93. The system of Clause 91 or 92, wherein the medical procedure is a patient scan.
Clause 94. The system of any of Clauses 80-93, wherein the remote source comprises a skilled user of the medical device.
Clause 95. The system of any of Clauses 80-94, wherein the remote source comprises an artificial intelligence software.
Clause 96. The system of Clause 91, wherein the second device being configured to permit the remote source to control an operation or attribute of the medical device allows the remote source to adjust an image taken by the medical device by adjusting a depth of focus, power, control the image features, pause the video feed, align the video, etc.
Clause 97. The system of Clause 91, wherein the second device being configured to permit the remote source to control an operation or attribute of the medical device allows the remote source to capture an image or video related to the medical procedure via the medical device.
Clause 98. The system of any of Clauses 80-97, wherein the medical device provides haptic feedback to its operator.
Clause 99. The system of Clause 98, wherein the remote source controls the haptic feedback to guide the operator of the medical device.
Clause 100. A method comprising any of the steps disclosed in the above Clauses.
Clause 101. A system comprising one or more devices configured to perform any of the methods disclosed in the above Clauses.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order and are not meant to be limited to the specific order or hierarchy presented.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Furthermore, to the extent that the term “include,” “have,” or the like is used in the description or the claims, such term is intended to be inclusive in a manner similar to the term “comprise” as “comprise” is interpreted when employed as a transitional word in a claim.

As used herein, the term “about” is relative to the actual value stated, as will be appreciated by those of skill in the art, and allows for approximations, inaccuracies and limits of measurement under the relevant circumstances. In one or more aspects, the terms “about,” “substantially,” and “approximately” may provide an industry-accepted tolerance for their corresponding terms and/or relativity between items, such as a tolerance of from less than one percent to ten percent of the actual value stated, and other suitable tolerances.

As used herein, the term “comprising” indicates the presence of the specified integer(s), but allows for the possibility of other integers, unspecified. This term does not imply any particular proportion of the specified integers. Variations of the word “comprising,” such as “comprise” and “comprises,” have correspondingly similar meanings.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

What is claimed is:

1-99. (canceled)

100. A method for providing remote guidance from a skilled resource to a local user during a medical procedure on a patient for enhancing the quality of the medical procedure performed by the local user, the method comprising:
transmitting information, representative of a procedure being performed by the local user, to the skilled resource, wherein the local user performs the procedure using a medical device;
permitting the skilled resource to transmit feedback to the local user; and
conveying the feedback to the local user via a communication interface.

101. The method of claim 100, wherein the permitting the skilled resource to transmit feedback to the local user comprises permitting the skilled resource to indicate at least one suggested position or movement of the medical device.

102. The method of claim 100, wherein the transmitting information comprises sending a video feed to the skilled resource of the local user performing the medical procedure or providing one or more images to the skilled resource.

103. The method of claim 100, wherein the permitting the skilled resource to transmit feedback comprises the skilled resource overlaying notation over the information transmitted to the skilled resource.

104. The method of claim 100, wherein the conveying the feedback comprises providing visual feedback to the local user using a first communication device or transmitting an audio instruction to the local user.

105. The method of claim 100, wherein the conveying the feedback comprises providing a graphic overlay of an example routine, target area on the patient, position of the medical device, or movement of the medical device.

106. The method of claim 100, wherein the skilled resource comprises a skilled user of the medical device or an artificial intelligence software.

107. The method of claim 100, further comprising permitting the skilled resource to transmit an example routine to the local user for viewing.

108. The method of claim 100, wherein the permitting the skilled resource to transmit feedback comprises permitting control of at least one attribute of the medical device by the skilled resource.

109. The method of claim 100, wherein the permitting the skilled resource to transmit feedback to the local user comprises permitting the skilled resource to mark a virtual target on the patient or annotate the video feed with a symbol, line, or highlight tool.

110. A method for enhancing a device operator's use of a medical device via feedback from a remote source during a medical procedure, the method comprising:
performing a medical procedure using the medical device;
transmitting a video, via a shared application, of the medical procedure from a video-enabled device of the device operator to a remote device of the remote source, the shared application permitting sharing and viewing of the video; and
permitting the remote source control of an operation or attribute of the medical device via the shared application.

111. The method of claim 110, further comprising permitting the remote source to transmit feedback to the device operator via (i) a notation overlaid onto the video transmitted to the remote source or (ii) a marking of a virtual target on a patient or annotate the video with a symbol, line, or highlight tool.

112. The method of claim 110, further comprising permitting the remote source to transmit an example routine to the device operator for viewing.

113. The method of claim 110, wherein the permitting the remote source control comprises permitting the remote source to change a mode of the medical device.

114. The method of claim 110, wherein the permitting the remote source control comprises, in response to a signal from the remote source, generating haptic feedback via the medical device to allow the remote source to communicate with the medical device operator.

115. A system for enhancing use of a medical device via feedback from a remote source during a medical procedure, the system comprising:
a medical device for performing a medical procedure;
a shared application software for communicating with the medical device;
a camera-enabled first device configured to run the shared application software and permit visual capture of the medical procedure;
a remote second device configured to run the shared application software and receive images or video of the medical procedure from the first device via the shared application, the second device being configured to permit the remote source to provide feedback based on performance of the medical procedure and to control an operation or attribute of the medical device via the shared application software for enhancing use of the device by a device operator.

116. The system of claim 115, wherein the second device being configured to permit the remote source to provide feedback allows the remote source to provide visual feedback using the remote second device.

117. The system of claim 116, wherein the visual feedback comprises a graphic overlay of an example routine, target area on a patient, position of the medical device, movement of the medical device, or an animation of at least one suggested position or movement of the medical device.

118. The system of claim 115, wherein the first device or the second device is a smartphone or a tablet.

119. The system of claim 115, wherein the remote source comprises a skilled user of the medical device or an artificial intelligence software.
